(19) United States
(12) Patent Application Publication
(10) Pub. No.: US 2022/0031896 A1
(43) Pub. Date: Feb. 3, 2022

(54) Disinfecting Tray

(71) Applicant: Jeffrey K. Ballard, Fleetwood, NC (US)

(72) Inventor: Jeffrey K. Ballard, Fleetwood, NC (US)

(21) Appl. No.: 16/947,421

(22) Filed: Jul. 31, 2020

(51) Int. Cl. A61L 2/26; A61L 2/18; A47L 23/26; B08B 3/08; B08B 7/04

(52) U.S. Cl. CPC A61L 2/26; A61L 2/186; A61L 2101/02; B08B 3/08; B08B 7/04; A47L 23/266

(57) ABSTRACT

A decontamination system with a water inlet to receive water from a water source, and a disinfectant inlet to receive disinfectant from a disinfectant source. A dispenser mixes the disinfectant with the water, according to a desired concentration, to produce a disinfectant solution. A supply valve releases a desired amount of the disinfectant solution. A reservoir with a substantially open top receives the desired amount of disinfectant solution. A mat within the reservoir is adapted to be stood upon by a user and remain substantially in place within the reservoir while the user scrubs the soles of shoes worn by the user against the mat. An overflow valve on a sidewall of the reservoir allows disinfectant solution within the reservoir to flow out of the reservoir when the disinfectant solution attains a desired depth within the reservoir.

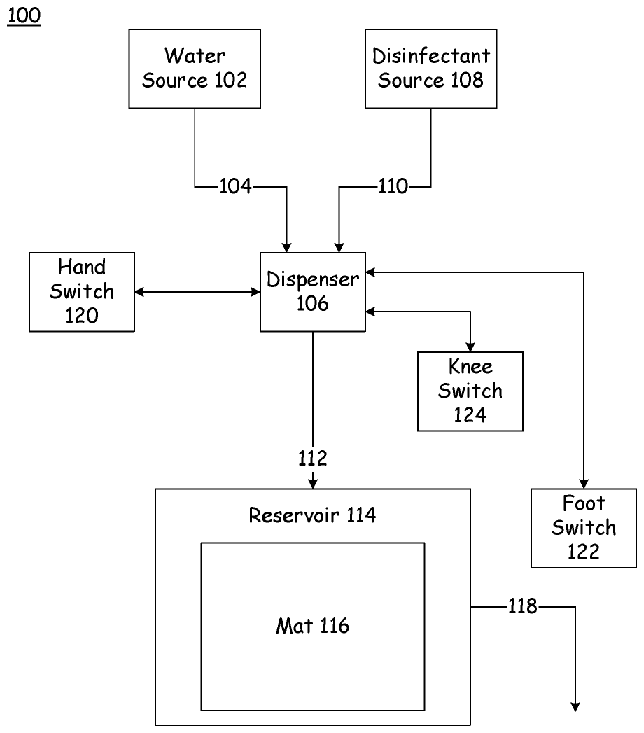

### FIELD

This invention relates to the field of cleaning products. More particularly, this invention relates to an apparatus for cleaning footwear, and most especially the soles of footwear.

### INTRODUCTION

Foot traffic is a major vector for the spread of contamination, including pathogens. Just over the course of a single day or a single shift at a place of business, the amount of contamination that is brought in through the door by pedestrian traffic can pose a cleanliness and health hazard to those in the home or business. Further, cleaning the tracked-in contamination can be time-consuming and expensive, and if not regularly maintained, ineffective.

What is needed, therefore, is a system that can tend to reduce issues such as those suggested by the description above, at least in part.

### SUMMARY

The above and other needs are met by a decontamination system with a water inlet to receive water from a water source, and a disinfectant inlet to receive disinfectant from a disinfectant source. A dispenser mixes the disinfectant with the water, according to a desired concentration, to produce a disinfectant solution. A supply valve releases a desired amount of the disinfectant solution. A reservoir with a substantially open top receives the desired amount of disinfectant solution. A mat within the reservoir is adapted to be stood upon by a user and remain substantially in place within the reservoir while the user scrubs the soles of shoes worn by the user against the mat. An overflow valve on a sidewall of the reservoir allows disinfectant solution within the reservoir to flow out of the reservoir when the disinfectant solution attains a desired depth within the reservoir.

In various embodiments according to this aspect of the invention, the disinfectant is hydrogen peroxide. In some embodiments, the mat is artificial turf. In some embodiments, the mat is artificial turf with perforations therein for the disinfectant solution to come up therethrough. In some embodiments, the supply valve is a manually-activated switch adapted to be selectively engaged by the user, which upon activation allows disinfectant solution to flow into the reservoir. In some embodiments, the switch is adapted to be engaged by hand and is disposed at a height that is substantially waist-high on the user. In some embodiments, the switch is adapted to be engaged by foot and is disposed at substantially ground level. In some embodiments, the switch is adapted to be engaged by knee, and is disposed at a height that is substantially midway between a floor and waist-height on the user. In some embodiments, the reservoir is a system having a substantially rectangular length and width, and a depth of no more than about two inches. In some embodiments, the overflow valve is a weir that allows disinfectant solution to flow out of the reservoir when the depth of the disinfectant solution is just above a top surface of the mat. In some embodiments, the mat is not fixed within the reservoir, but can be selectively removed and replaced when worn. In some embodiments, the reservoir is formed of a thermoplastic resin. In some embodiments, the reservoir is formed of metal.

### DRAWINGS

Further advantages of the invention are apparent by reference to the detailed description when considered in conjunction with the FIGURE, which is not to scale so as to more clearly show the details, and which is a functional block diagram of a decontamination system according to an embodiment of the present invention.

### DESCRIPTION

With reference now to the FIGURE, there is depicted a decontamination system 100 according to an embodiment of the present invention. The system 100 includes a water inlet 104 that receives water from a water source 102. In various embodiments, the water source 102 is a fixed-line input, such as a copper line or a flexible line running from a plumping system in the house or building. In other embodiments the water source 102 is a more temporary source, such as a garden hose or other quickly-disconnected line.

The system 100 also includes a disinfectant inlet 110 that receives disinfectant from a disinfectant source 108. In some embodiments the disinfectant is a concentrated solution, such as hydrogen peroxide, or chlorine. The water and the disinfectant are combined in a dispenser 106. In some embodiments, the concentrated disinfectant source 108 is provided within a package that is disposed within the dispenser 106, and is replaced from time to time as it is depleted. In some embodiments the dispenser 106 includes an adjustment for setting the desired concentration of disinfectant, while in other embodiments the desired dilution is set and cannot be changed. In some embodiments, the desired concentration is about 0.047% by volume of hydrogen peroxide, which is achieved by a dilution rate of about six ounces of concentrated disinfectant per gallon of water.

The dispenser 106 provides the disinfectant solution to a reservoir 114 through a supply valve 112. In some embodiments the supply valve 112 is controlled by one or more switches, such as a switch 120 that is disposed at a height that is convenient to be actuated by hand, such as about at waist height or so of a user, or a switch 122 that is disposed at a height that is convenient to be actuated by foot, such as a few inches up on a wall or on the floor, or a switch 124 that is disposed at a height that is convenient to be actuated by knee, such as midway between the floor and about waist-height on the user. When one or more of the switches 120, 122, and 124 are activated, either a given amount of the disinfectant solution is dispensed into the reservoir 114, or the disinfectant solution is dispensed for the length of time that the switch 120, 122, or 124 is activated.

The reservoir 114 in some embodiments is formed of a material that is at least relatively resistant to chemical attack or corrosion by the disinfectant solution. In some embodiments the reservoir 114 is formed of plastic or metal, or a combination of the two. For example, in one embodiment the reservoir 114 is formed of metal with a liner of some type that is resistant to the disinfectant solution.

In some embodiments the reservoir 114 is in the basic form of a tray, within four sides and a bottom but a top that is open. In some embodiments the length and width of the reservoir 114 is large enough so that a user can stand with both feet inside of the reservoir 114, and have room to shuffle or otherwise move his feet so as to clean his feet in the disinfectant solution. In some embodiments the reservoir 114 is about two feet square. The reservoir 114 in some embodiments can be much larger, with multiple inlets 110 and switches 120, 122, or 124, so as to accommodate several users at one time.

Disposed with the reservoir 114 is a mat 116 that has a rough upper surface, such that it provides some degree of friction between the mat 116 and the soles of the user's shoes. In some embodiments that mat 116 is formed of a material such as artificial turf, indoor-outdoor carpet, SCOTCH-BRITE®, or some other material that is specially manufactured to provide the properties as described herein. In some embodiments, the underside of the mat 116 is formed of a material that offers at least some amount of resistance to sliding of the mat 116 against the bottom of the reservoir 114 while the decontamination system 100 is in use. In some embodiments the matt 116 is formed of one or more materials that are at least somewhat resistant to chemical attack or other degradation arising from being substantially submerged in the disinfectant solution.

In some embodiments the mat 116 lies on the bottom surface of the reservoir 114, and has perforations that allow the disinfectant solution to permeate to different portions of the mat 116, and otherwise circulate through the reservoir 114. In this manner, the solution does not become unduly contaminated in one area of the reservoir 114, while remaining relatively unused in another area of the reservoir 114. In some embodiments that mat 116 is selectively removable from the reservoir 114, so that the mat 116 can be replaced when it becomes worn, damaged, or contaminated.

In some embodiments the sides of the reservoir 114 are sufficiently high so as to retain a desired amount of solution within the reservoir 114 without it splashing too much out of the reservoir 114 as it is used to clean the soles of the user's shoes. However, it is also desired that the sides of the reservoir 114 not be so high as to present an undue tripping hazard to the user. In some embodiments the sides of the reservoir 114 are about an inch and a half in height, or no more than about two inches in height. In some embodiments, the sides of the reservoir 114 are flared at the top.

The reservoir 114 includes an overflow valve 118 that allows the disinfectant solution to flow out of the reservoir 114 upon the occurrence of one or more different events. In one embodiment, the overflow valve 118 can be actuated to drain all of the solution out of the reservoir 114, either upon manual activation, such as when the disinfectant solution appears to be dirty, or automatically, such as upon a given time schedule. In another embodiment, the overflow valve 118 is formed as a weir, such as a port in the side of the reservoir 114 that connected to a tube that allows disinfectant solution to drain out of the reservoir 114 when the solution reaches a certain height within the reservoir 114. In some embodiments, the overflow valve 118 is positioned so as to allow the disinfectant solution to flow out of the reservoir 114 whenever the depth of the disinfectant solution within the reservoir 114 is greater than a depth that is just sufficient to cover the top of the mat 116.

The decontamination system 100 is used by the user standing inside the reservoir 114 with his feet on the mat 116. The user presses one or more of the switches 120, 122, and 124 to allow fresh solution to flow into the reservoir 114. This can be done either before or after the user steps into the reservoir 114, as needed or desired. The user then shuffles or scrubs his feet back and forth on the mat 116 within the reservoir 114. The overflow valve 118 maintains the level of the disinfectant solution such that the depth of the disinfectant solution is sufficient to wet the soles of the user's shoes, but not get the rest of his shoes or clothing unduly wet. The user can also activate the hand switch 120 during use of the system 100, so as to refresh the disinfectant solution during his use of the decontamination system 100. When the user has spent a sufficient length of time using the system 100, or has achieved some other type of use goal, the user steps out of the reservoir 114 and continues along his way.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A decontamination system, comprising:
a water inlet adapted to receive water from a water source,
a disinfectant inlet adapted to receive disinfectant from a disinfectant source,
a dispenser adapted to mix the disinfectant with the water, according to a desired concentration, to produce a disinfectant solution,
a supply valve adapted to release a desired amount of the disinfectant solution,
a reservoir having a substantially open top and adapted to receive the desired amount of disinfectant solution,
a mat disposed within the reservoir and adapted to be stood upon by a user and remain substantially in place within the reservoir while the user scrubs the soles of shoes worn by the user against the mat, and
an overflow valve disposed on a sidewall of the reservoir and adapted to allow disinfectant solution within the reservoir to flow out of the reservoir when the disinfectant solution attains a desired depth within the reservoir.

2. The decontamination system of claim 1, wherein the disinfectant comprises hydrogen peroxide.

3. The decontamination system of claim 1, wherein the mat comprises artificial turf.

4. The decontamination system of claim 1, wherein the mat comprises artificial turf having perforations therein for the disinfectant solution to come up therethrough.

5. The decontamination system of claim 1, wherein the supply valve comprises a manually-activated switch adapted to be selectively engaged by the user, which upon activation allows disinfectant solution to flow into the reservoir.

6. The decontamination system of claim 5, wherein the switch is adapted to be engaged by hand and is disposed at a height that is substantially waist-high on the user.

7. The decontamination system of claim 5, wherein the switch is adapted to be engaged by foot and is disposed at substantially ground level.

8. The decontamination system of claim 1, wherein the reservoir comprises a system having a substantially rectangular length and width, and a depth of no more than about two inches.

9. The decontamination system of claim 1, wherein the overflow valve is a weir that allows disinfectant solution to flow out of the reservoir when the depth of the disinfectant solution is just above a top surface of the mat.

10. The decontamination system of claim 1, wherein the mat is not fixed within the reservoir, but can be selectively removed and replaced when worn.

11. The decontamination system of claim 1, wherein the reservoir is formed of a thermoplastic resin.

12. The decontamination system of claim 1, wherein the reservoir is formed of metal.

13. A decontamination system, comprising:
a water inlet adapted to receive water from a water source,
a disinfectant inlet adapted to receive disinfectant from a disinfectant source,
a dispenser adapted to mix the disinfectant with the water, according to a desired concentration, to produce a disinfectant solution,
a manually-activated switch adapted to be selectively engaged by the user to release a desired amount of the disinfectant solution,
a reservoir forming a system with a substantially rectangular length and width, a depth of no more than about two inches, and a substantially open top and adapted to receive the desired amount of disinfectant solution,
a mat of perforated artificial turf disposed within the reservoir and adapted to be stood upon by a user and remain substantially in place within the reservoir while the user scrubs the soles of shoes worn by the user against the mat, and
an overflow valve disposed on a sidewall of the reservoir and adapted to allow disinfectant solution within the reservoir to flow out of the reservoir when the disinfectant solution attains a desired depth within the reservoir.

14. The decontamination system of claim 13, wherein the disinfectant comprises hydrogen peroxide.

15. The decontamination system of claim 13, wherein the switch is adapted to be engaged by hand and is disposed at a height that is substantially waist-high on the user.

16. The decontamination system of claim 13, wherein the switch is adapted to be engaged by foot and is disposed at substantially ground level.

17. The decontamination system of claim 13, wherein the overflow valve is a weir that allows disinfectant solution to flow out of the reservoir when the depth of the disinfectant solution is just above a top surface of the mat.

18. The decontamination system of claim 13, wherein the mat is not fixed within the reservoir, but can be selectively removed and replaced when worn.

19. The decontamination system of claim 13, wherein the reservoir is formed of a thermoplastic resin.

20. The decontamination system of claim 13, wherein the reservoir is formed of metal.
